(19) United States
(12) Patent Application Publication
(10) Pub. No.: US 2003/0055084 A1
(43) Pub. Date: Mar. 20, 2003

(54) Pharmaceutical compounds and methods of use thereof

(75) Inventors: Gurmit Grewal, Natick, MA (US); Ralph Scannell, Hopkinton, MA (US); Xiong Cai, Belmont, MA (US); Michelle Young, Belmont, MA (US); Aberra Fura, Lawerenceville, NJ (US)

Correspondence Address: EDWARDS & ANGELL, LLP., P.O. BOX 9169, BOSTON, MA, 02209, US

(73) Assignee: Millennium Pharmaceutical, Inc.

(21) Appl. No.: 10/209,188

(22) Filed: Jul. 30, 2002

Related U.S. Application Data
(60) Provisional application No. 60/308,945, filed on Jul. 30, 2001.

(51) Int. Cl. A61K 31/453; A61K 31/451; A61K 31/4025; A61K 31/381; A61K 31/343; C 07D 4 9/02; C 07D 4 5/02

(52) U.S. Cl. 514/320; 514/422; 514/438; 514/443; 514/444; 514/469; 546/202; 546/196; 548/525; 549/49; 549/60; 549/462

(57) ABSTRACT

The present invention provides substituted benzofuran, indene, thianaphthene and oxidized thianaphthene compounds and methods of treatment and pharmaceutical compositions that comprise such compounds. Preferred compounds of the invention contain benzofuran, indene or thianaphthene group substituted with a tetrahydrofuran or other alicyclic group.

This application claims the benefit of U.S. Provisional Application Serial No. 60/308,945, filed Jul. 30, 2001, the teachings of which are incorporated herein by reference.

### BACKGROUND

1. Field of the Invention

The present invention provides substituted benzofuran, indene, thianaphthene and oxidized thianaphthene compounds and methods of treatment and pharmaceutical compositions that comprise such compounds. Preferred compounds of the invention contain benzofuran, indene or thianaphthene group substituted with a tetrahydrofuran or other alicyclic group.

2. Background

Leukotrienes are recognized potent local mediators, playing a significant role in inflammatory and allegeric responses, including arthritis, asthma, psoriasis and thrombotic disease. Leukotrienes are produced by the oxidation of arachidonic acid by lipoxygenase. More particularly, arachidonic acid is oxidized by 5-lipooxygenase to the hydroperoxide, 5-hydroperoxy-eicosatetraenoic acid (5-HPETE), that is converted to leukotriene A₄, that in turn can be converted to leukotriene B₄, C₄, or D₄. The slow-reacting substance of anaphylaxis is now known to be a mixture of leukotrienes C₄, D₄ and E₄, all of which are potent bronchoconstrictors.

Efforts have been made to identify receptor antagonists or inhibitors of leukotriene biosynthesis, to prevent or minimize pathogenic inflammatory responses mediated by leukotrienes. For example, European Patent Application Nos. 901171171.0 and 901170171.0 report indole, benzofuran, and benzothiophene lipoxygenase inhibiting compounds. Various 2,5-disubstituted tetrahydrothiophenes and pyrrolidines have exhibited significant biological activity, including as lipoxygenase inhibitors. See U.S. Pat. Nos. 5,703,093; 5,681,966; 5,648,486; 5,434,151; and 5,358,938.

### SUMMARY OF THE INVENTION

The invention provides new substituted benzofuran, indene, thianaphthene and oxidized thianaphthene compounds. Compounds of the invention are useful for a variety of therapeutic applications, including treatment of a mammal that is suffering from or susceptible to immune, allergic and cardiovascular disorders and diseases.

More particularly, preferred compounds of the invention include those of the following Formula I:

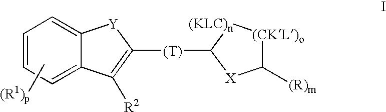

wherein, in Formula I, each R, R¹, R², K, L, K′, and L′ is independently hydrogen or a non-hydrogen substituent such as halogen, cyano, hydroxyl, optionally substituted alkyl preferably having 1 to about 20 carbon atoms, optionally substituted alkenyl preferably having 2 to about 20 carbon atoms, optionally substituted alkynyl preferably having 2 to about 20 carbon atoms, optionally substituted alkoxy preferably having 1 to about 20 carbon atoms, optionally substituted alkylthio preferably having 1 to about 20 carbon atoms, optionally substituted alkylsulfinyl preferably having 1 to about 20 carbon atoms, optionally substituted alkylsulfonyl preferably having 1 to about 20 carbon atoms, optionally substituted aminoalkyl preferably having about 1 to about 20 carbon atoms, optionally substituted alkanoyl preferably having 1 to about 20 carbon atoms, optionally substituted carbocyclic aryl, optionally substituted aralkyl;

X is O, S, S(O), S(O)₂, NH, substituted N or a chemical bond;

T is a chemical bond, optionally substituted alkylene, optionally substituted alkenylene, optionally substituted alkynylene, optionally substituted heteroalkynylene, or a hetero atoms such as O, S, S(O), S(O)₂, or NR wherein R is the same as defined immediately above;

Y is O, S, S(O), S(O)₂ or a chemical bond;

m is 0 (where the α ring position is hydrogen-substituted), 1 or 2;

o and n are each integers of 0 or greater, the sum of o and n being from 1 to about 8, preferably the sum of o and n being from 2 to about 5;

p is an integer of from 0 (where the available ring positions are hydrogen-substituted) to 4, preferably 1, 2 or 3; and pharmaceutically acceptable salts thereof.

Preferred compounds of the invention include those where R includes one or more hetero atoms (particularly N or S), such as compounds of the following Formula II and pharmaceutically acceptable salts thereof:

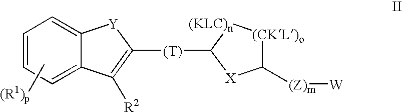

wherein, in Formula II, R¹, R², Y, T, X, K, L, K′, L′n, o, and p are each the same as defined in Formula I above;

Z is O, S, S(O), S(O)₂, NR¹ or a chemical bond;

W is —AN(OM)C(O)N(R¹R²), —N(OM)C(O)N(R¹R²), —AN(R)C(O)N(OM)R¹, —N(R)C(O)N(OM)R¹, —AN(OM)C(O)R¹, —N(OM)C(O)R¹, —AC(O)N(OM)R¹, —C(O)N(OM)R¹, —C(O)NHA, where A is optionally substituted alkyl, optionally substituted alkenyl, optionally substituted alkynyl, optionally substituted carbocyclic aryl, and where one or more carbon atoms can be optionally replaced with O, substituted O, N, substituted N, S, or substituted S; and M is hydrogen, a pharmaceutically acceptable cation, or a metabolically cleavable leaving group;

m is 0 or 1.

Generally preferred compounds of the invention include comprise a benzofuran group, such as compounds of the following Formula III:

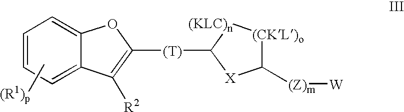

wherein, in Formula III, R¹, R², T, K, L, K′L′, X, Z, W, m, n, o, and p are the same as defined in Formula II; and pharmaceutically acceptable salts thereof.

Particularly preferred compounds of the invention include those that comprise a tetrahydrofuran group directly substituted with an optionally substituted benzofuran, particularly 2,5-substituted tetrahydrofurans such as those of the following Formula IV and pharmaceutically acceptable salts thereof:

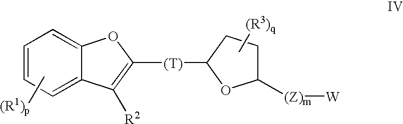

wherein, in Formula IV, R¹, R², X, Z, W, m, and p are the same as defined in Formula II; R³ is hydrogen or a non-hydrogen substituent, e.g. selected from the same group as defined for R¹ and R²; and q is an integer of from 0 (i.e. where the depicted 3,4-alicyclic positions are each —CH₂—), 1, 2, 3 or 4, and preferably q is 0, 1 or 2.

Preferred compounds of the invention also include those that comprise a thianaphthene group, such as compounds of the following Formula V and pharmaceutically acceptable salts thereof:

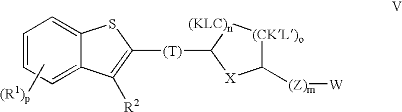

wherein, in Formula V, R¹, R², T, K, L, K′, L′, X, Z, W, m, n, o, and p are each the same as defined in Formula III above.

Particularly preferred compounds of the invention include those that comprise a tetrahydrofuran group directly substituted with an optionally substituted thianaphthene group, particularly 2,5-substituted tetrahydrofurans such as those of the following Formula VI and pharmaceutically acceptable salts thereof:

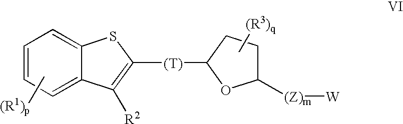

wherein, in Formula VI, R¹, R², R³, Z, W, m, p and q are each the same as defined in Formula IV above.

Preferred R¹ groups of compounds of the above Formula I through VI include hydrogen, halogen particularly F, Cl and Br; optionally substituted alkyl, particularly C₁₋₆alkyl that is optionally substituted by fluoro or other halogen and the like, such as trifluoromethyl; optionally substituted alkoxy, particularly C₁₋₆alkoxy optionally substituted by fluoro and other halogen and the like; optionally substituted carboxylic aryloxy such as optionally substituted phenoxy; and the like. Particularly preferred R¹ groups include 4-trifluoromethyl, 4-halo such as 4-fluoro, and 4-alkoxy such as 4-methoxy and 4-ethoxy, all where p is 1 (i.e. a single R¹ group). A chemical bond is a preferred T and Z group. Preferred compounds of the invention include those where K, L, K′, L′ and R³ are hydrogen. Preferred R and W groups include optionally substituted alkyl, particularly C₁₋₁₂ alkyl, such as a branched alkyl group, e.g. —(CH₂)ₙCH(C₁₋₆alkyl)H—, wherein n is 1-5, and specifically —(CH₂)₂CH(CH₃)H—, or lower alkynyl such as of the formula —C≡C—CH(C₁₋₆alkyl)-, including —C≡C—CH(CH₃)—.

In some instances, particularly for therapeutic applications, a selected stereoisomer of a compound of the above formulae may be preferred, e.g. where the selected stereoisomer is present in an amount of at least about 70 or 80 mole percent relative to other stereoisomer(s) of the compounds, more preferably where the selected stereoisomer is present in an amount of at least about 85, 90, 95, 97 or 99 mole percent relative to other stereoisomer(s) of the compound.

Specifically preferred compounds of the invention includes N-(4-(-5-[5-(trifluoromethyl)benzo[d]furan-2-yl]oxolan-2-yl)but-3-ynyl) amino-N-hydroxyamide and pharmaceutically acceptable salts thereof, and enantiomerically enriched mixtures thereof, particularly a mixture containing predominately N-(4-((2S,5S)-5-[5-(trifluoromethyl)benzo[d]furan-2-yl]oxolan-2-yl)but-3-ynyl) amino-N-hydroxyamide and pharmaceutically acceptable salts thereof as well as mixtures that contain predominately N-(4-((2S,5R)-5-[5-(trifluoromethyl)benzo[d]furan-2-yl]oxolan-2-yl)but-3-ynyl) amino-N-hydroxyamide and pharmaceutically acceptable salts thereof.

As mentioned above, compounds produced by the methods of the invention will be useful as pharmaceutical agents, including treatment of disorders or diseases mediated by 5-lipoxygenase. Compounds of the invention may be administered to a patient, particularly a mammal such as a human or other primate, to treat immune, allegeric and cardiovascular disorders and diseases, e.g. general inflammation, hypertension, skeletal-muscular disorders, osteoarthritis, gout, asthma, lung edema, adult respiratory distress syndrome, pain, aggregation of platelets, shock, rheumatoid arthritis, psoriatic arthritis, psoriasis, inflammatory bowel disease, chronic obstructive pulmonary disease (COPD), autoimmune uveitis, allergic encephalomyelitis, systemic lupus erythematosis, acute necrotizing hemmorrhagic encephalopathy, idiopathic thrombocytopenia, polychondritis, chronic active hepatitis, idiopathic sprue, Crohn's disease, Graves ophthalmopathy, primary biliary cirrhosis, uveitis posterior, interstitial lung fibrosis, allergic asthma and inappropriate allergic responses to environmental stimuli.

The invention also includes pharmaceutical compositions that comprise one or more compounds of Formula I and a pharmaceutically acceptable carrier.

Other aspects of the invention are disclosed infra.

### DETAILED DESCRIPTION OF THE INVENTION

As discussed above, the invention provides compounds of the following Formulae I through VI:

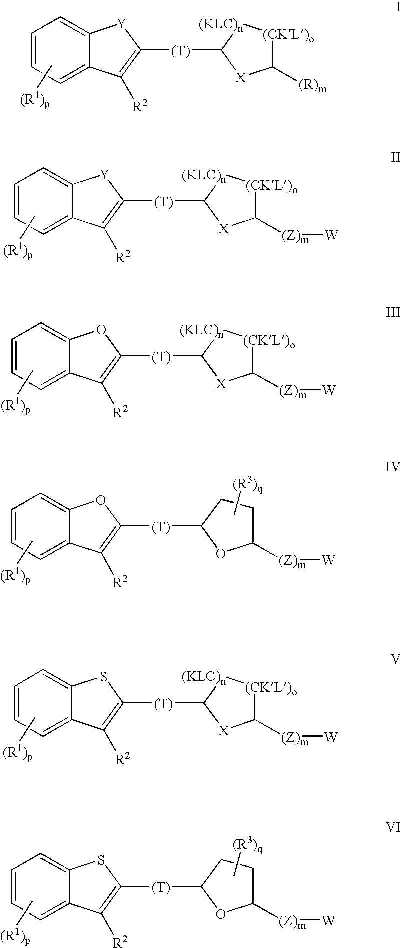

wherein R, R¹, R², R³, K, L, K′, L′, T, X, Y, Z and W, m, n, o, p and q are as defined above; and pharmaceutically acceptable salts thereof.

Particularly preferred compounds of the invention include those of the following structure 1 and pharmaceutically acceptable salts thereof:

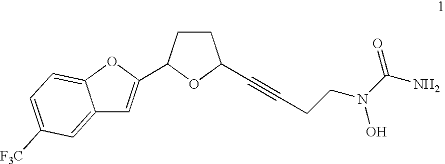

Optically active stereoisomers of compound 1 are especially preferred, such as compounds of the following structures 1A (the 2S,5S enantiomer) and 1B (the 2S,5R enantiomer) and pharmaceutically acceptable salts thereof:

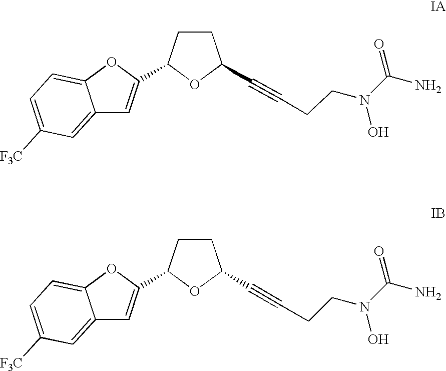

Additional specifically preferred compounds of the invention include the following and pharmaceutically acceptable salts thereof, and stereoisomers thereof:

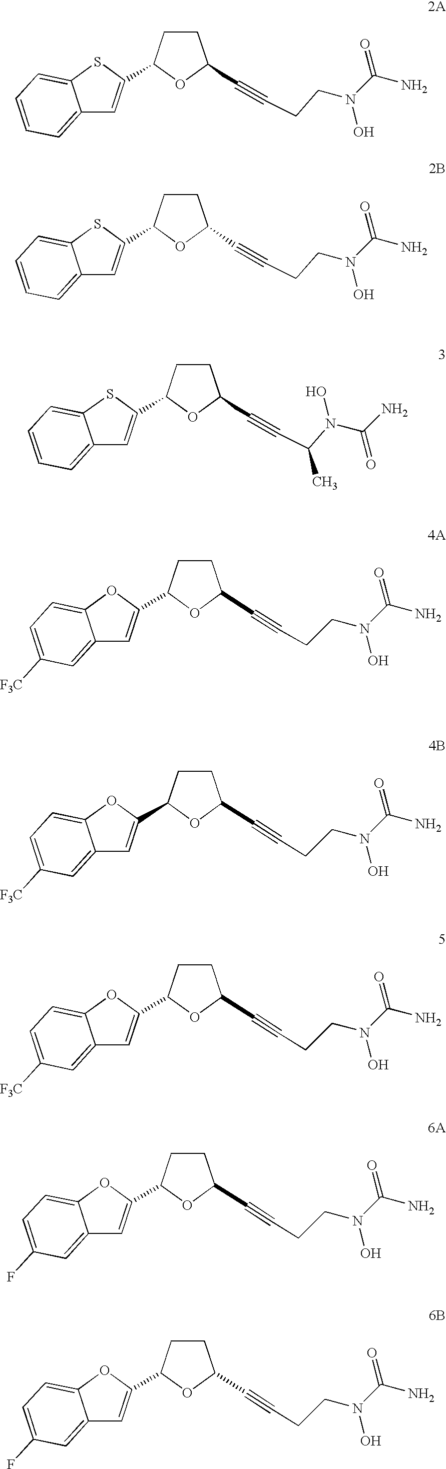

The term alkyl, as used herein, unless otherwise specified, refers to a saturated straight, branched, or cyclic hydrocarbon preferably of C₁ to C₁₂, more typically C₁ to C₆ saturated straight, branched, or cyclic (in the case of C₅₋₆) hydrocarbon, and specifically includes methyl, ethyl, propyl, isopropyl, butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl, pentyl, cyclopentyl, isopentyl, neopentyl, hexyl, isohexyl, cyclohexyl, and 3-methylpentyl. The alkyl group can be optionally substituted with any appropriate group, including but not limited to one or more moieties selected from the group consisting of halo, hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as disclosed in Greene et al., “Protective Groups in Organic Synthesis”, John Wiley and Sons, Second Edition, 1991.

The term halo, as used herein, refers to chloro, fluoro, iodo, or bromo.

The term alkenyl, as referred to herein, and unless otherwise specified, refers to a straight, branched, or cyclic (such as in the case of C₅₋₈, more typically C₅₋₆) hydrocarbon preferably of C₂ to C₁₂ with at least one double bond, optionally substituted as described above. More typically, an alkenyl group will have from 2 to 6 carbon atoms, and includes vinyl and allyl.

The term alkylamino refers to an amino group that has one or two optionally substituted alkyl, preferably one or two C₁₋₆alkyl groups.

The term alkynyl, as referred to herein, and unless otherwise specified, refers to preferably C₂ to C₁₂ straight or branched hydrocarbon with at least one triple bond, optionally substituted as described above. More typically, an alkynyl group will have from 2 to 6 carbon atoms, and includes acetylenyl, propynyl, and —C≡C—CH(C₁₋₆alkyl)-, including —C≡C—CH(CH₃)—.

The term carbocyclic aryl, as used herein, and unless otherwise specified, refers to non-hetero aromatic groups that have 1 to 3 separate or fused rings and 6 to about 18 carbon ring atoms and include e.g. phenyl, naphthyl, biphenyl, phenanthryl, anthracyl, and the like. The carbocyclic aryl group can be optionally substituted with any suitable group, including but not limited to one or moieties selected from the group consisting of halo, hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene et al., “Protective Groups in Organic Synthesis”, John Wiley and Sons, Second Edition, 1991, and preferably with halo (including but not limited to fluoro), lower alkoxy (including methoxy), lower aryloxy (including phenoxy), W, cyano, or R³.

The term haloalkyl, haloalkenyl, or haloalkynyl refers to alkyl, alkenyl, or alkynyl group in which at least one of the hydrogens in the group has been replaced with a halogen atom.

The term heteroaryl, heterocycle or heteroaromatic, as used herein, refers to an aromatic moiety that includes at least one sulfur, oxygen, or nitrogen in the aromatic ring, which can optionally be substituted as described above for the aryl groups. Non-limiting examples are pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, benzofuran, benzothienyl, isobenzofuryl, pyrazolyl, indolyl, purinyl, carbazolyl, benzimidazolyl, and isoxazolyl. Suitable heteroaromatic or heteroaryl groups will have 1 to 3 rings, 3 to 8 ring members in each ring and from 1 to 3 hetero atoms (N, O or S).

The term arylalkyl refers to a carbocyclic aryl group with an alkyl substituent.

The term alkylaryl refers to an alkyl group that has a carbocyclic aryl substituent.

The term organic or inorganic anion refers to an organic or inorganic moiety that carries a negative charge and can be used as the negative portion of a salt.

The term “pharmaceutically acceptable cation” refers to an organic or inorganic moiety that carries a positive charge and that can be administered in association with a pharmaceutical agent, for example, as a counter cation in a salt. Pharmaceutically acceptable cations are known to those of skill in the art, and include but are not limited to sodium, potassium, and quaternary amine.

The term “metabolically cleavable leaving group” refers to a moiety that can be cleaved in vivo from the molecule to which it is attached, and includes but it not limited to an organic or inorganic anion, a pharmaceutically acceptable cation, acryl (for example (alkyl)C(O), including acetyl, propionyl, and butyryl), alkyl, phosphate, sulfate and sulfonate.

Alkylene and heteroalkylene groups typically will have about 1 to about 8 atoms in the chain, more typically 1 to about 6 atoms in the linkage. Alkenylene, heteroalkenylene, alkynylene and heteroalkynylene groups typically will have about 2 to about 8 atoms in the chain, more typically 2 to about 6 atoms in the linkage, and one or more unsaturated carbon-carbon bonds, typically one or two unsaturated carbon-carbon bonds. A heteroalkylene, heteroalkenylene or heteroalkynylene group will have at least one hetero atom (N, O or S) as a divalent chain member.

The term alkanoyl refers to groups that in general formulae generally will have from 1 to about 16 carbon atoms and at least one carbonyl (C═O) moiety, more typically from 1 to about 8 carbon atoms, still more typically 1 to about 4-6 carbon atoms. The term alkylthio generally refers to moieties having one or more thioether linkages and preferably from 1 to about 12 carbon atoms, more preferably from 1 to about 6 carbon atoms. The term alkylsulfinyl generally refers to moieties having one or more sulfinyl (S(O)) linkages and preferably from 1 to about 12 carbon atoms, more preferably from 1 to about 6 carbon atoms. The term alkylsulfonyl generally refers to moieties having one or more sulfonyl (S(O)₂) linkages and preferably from 1 to about 12 carbon atoms, more preferably from 1 to about 6 carbon atoms. The term aminoalkyl generally refers to groups having one or more N atoms and from 1 to about 12 carbon atoms, preferably from 1 to about 6 carbon atoms.

As discussed above, various substituent groups of the above formulae may be optionally substituted. Suitable groups that may be present on such a “substituted” group include e.g. halogen such as fluoro, chloro, bromo and iodo; cyano; hydroxyl; nitro; azido; sulfhydryl; alkanoyl e.g. C₁₋₆ alkanoyl group such as acetyl and the like; carboxamido; alkyl groups including those groups having 1 to about 12 carbon atoms, preferably from 1 to about 6 carbon atoms; alkenyl and alkynyl groups including groups having one or more unsaturated linkages and from 2 to about 12 carbon atoms, preferably from 2 to about 6 carbon atoms; alkoxy groups having one or more oxygen linkages and from 1 to about 12 carbon atoms, preferably 1 to about 6 carbon atoms; aryloxy such as phenoxy; alkylthio groups including those moieties having one or more thioether linkages and from 1 to about 12 carbon atoms, preferably from 1 to about 6 carbon atoms; alkylsulfinyl groups including those moieties having one or more sulfinyl linkages and from 1 to about 12 carbon atoms, preferably from 1 to about 6 carbon atoms; alkylsulfonyl groups including those moieties having one or more sulfonyl linkages and from 1 to about 12 carbon atoms, preferably from 1 to about 6 carbon atoms; aminoalkyl groups such as groups having one or more N atoms and from 1 to about 12 carbon atoms, preferably from 1 to about 6 carbon atoms; carbocyclic aryl having 6 or more carbons, particularly phenyl; aryloxy such as phenoxy; aralkyl having 1 to 3 separate or fused rings and from 6 to about 18 carbon ring atoms, with benzyl being a preferred group; aralkoxy having 1 to 3 separate or fused rings and from 6 to about 18 carbon ring atoms, with O-benzyl being a preferred group; or a heteroaromatic or heteroalicyclic group having 1 to 3 separate or fused rings with 3 to about 8 members per ring and one or more N, O or S atoms, e.g. coumarinyl, quinolinyl, pyridyl, pyrazinyl, pyrimidyl, furyl, pyrrolyl, thienyl, thiazolyl, oxazolyl, imidazolyl, indolyl, benzofuranyl, benzothiazolyl, tetrahydrofuranyl, tetrahydropyranyl, piperidinyl, morpholino and pyrrolidinyl. A “substituted” group of a compound of the invention prepared by a method of the invention may be substituted at one or more available positions, typically 1 to about 3 positions, by one or more suitable groups such as those listed immediately above.

Compounds of the invention may be readily prepared, including by procedures disclosed in U.S. Pat. No. 6,025,384 and PCT Published Applications WO 00001683A1; WO 000016701A1; and WO 00001381A1. More specifically, compounds of the invention can be prepared by the general procedures shown in the following Scheme 1, which depicts exemplifies synthesis of Compounds 1A and 1B. It will be understood that a variety of other compounds can be employed in a similar manner as described below with respect to the exemplified compounds. For instance, the carbocyclic aryl group of 4-trifluoromethylphenol is depicted throughout Scheme 1, although a wide variety of other aryl groups could be employed in the same or similar manner as fluorophenyl, particularly other substituted phenols to provide other benzofuran compounds, or a substituted or unsubstituted benzeone thiol to provide a thianaphthene compound. Additionally while compounds in the below Scheme 1 depict substitution at the ring carbons α to the ring hetero atom, other ring positions can be readily substituted e.g. by using appropriately substituted starting reagents. Also, while various stereoisomers are depicted in the below Scheme 1, corresponding other stereoisomers and diastereomers can be readily obtained by use of the corresponding optically active reagents or enantioselective reactions or separations.

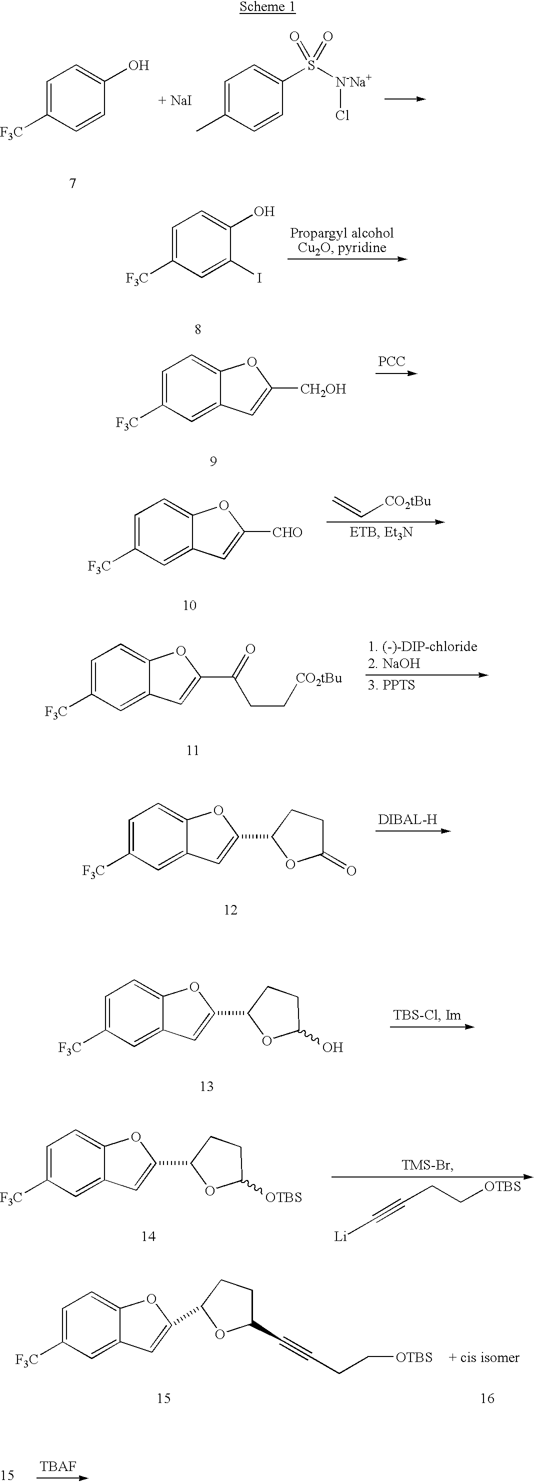

The above Scheme 1 exemplifies a preferred preparative method of the invention wherein phenolic compound 7 is reacted in the presence of base such as the base prepared from sodium hydride and cloramine-T to provide 2-iodo compound 8 which forms benzofuran alcohol 9 upon treatment with propargyl alcohol in the presence of a suitable catalyst such as a copper catalyst. Oxidation of the benzofuran alcohol 9 provides the α,β-unsaturated aldehyde 10 which can react with an acrylate in the presence of base to provide the keto ester 11. The substituted benzofuran 11 is cyclized to provide the substituted γ-butyrolactone 12. Preferably, the cyclization is conducted in the presence of an optically active reagent to provide an enanomerically-enriched mixture of a lactone, as exemplified by 12.

Lactone 12 is then reduced to the hydroxy-tetrahydrofuran 13 with a suitable reducing agent, preferably a metal hydride such as DIBAL-H and the like. The hydroxy substituent of the tetrahydrofuran 7 is then preferably protected e.g. as an ester or ether. Thus, as depicted in the above Scheme, the hydroxy moiety of 13 can be reacted with a suitable silyl reagent, e.g. to form the t-butyldimethylsilyl ether 14, or with an esterification reagent, e.g. an anhydride such as acetic anhydride to provide an acetyl ester. The protected substituted tetrahydrofuran then can be reacted to provide further substitution e.g. with a nucleophile such as a 1-alkynyl reagent as depicted in the Scheme in the presence of a strong base. The resulting compound 15 may be further functionalized after deprotection as desired e.g. by amidation using an N,O-substituted hydroxylamine in the presence of dehydrating reagents such as triphenylphosphine and diisopropylazodicarboxlate, followed by treating intermediate 18 with ammonia to yield preferred Compound 1A. Compound 1B can be provided by similar reaction of stereoisomer 16 as generally shown in the above Scheme.

Compounds of the invention that have substituted sulfur alicyclic ring members (e.g. compounds of Formulae I, II, IV or V wherein X or Y is —S(O)—, —S(O)₂—) can be readily prepared. For example, the prepared thio alicyclic group can be oxidized to provide a ring member of —S(O)— or —S(O)₂— by known techniques such as with H₂O₂ and/or sodium periodate.

As discussed above, compounds of the invention are useful for numerous therapeutic applications. The compounds can be administered to a subject, particularly a mammal such as human, in need of treatment, by a variety of routes. For example, the compound can be administered orally, parenterally, intravenously, intradermally, subcutaneously, or topically.

The active compound may be administered to a subject as a pharmaceutically active salt, e.g. salts formed by addition of an inorganic acid such as hydrochloric acid, hydrobromic acid, phosphoric acid, etc., or an organic acid such as acetic acid, oxalic acid, tartaric acid, succinic acid, etc. Base addition salts also can be formulated if an appropriate acidic group is present on the compound. For example, suitable base addition salts include those formed by addition of metal cations such as zinc, calcium, etc., or salts formed by addition of ammonium, tetraethylammonium, etc.

For example, compounds of the invention can be employed, either alone or in combination with one or more other therapeutic agents, as a pharmaceutical composition in mixture with a conventional excipient, i.e. pharmaceutically acceptable organic or inorganic carrier substances suitable e.g. for parenteral, enteral or intranasal application which do not deleteriously react with active compounds and are not deleterious to the recipient thereof. Suitable pharmaceutically acceptable carrier include but are not limited to water, salt solutions, alcohol, vegetable oils, polyethylene glycols, gelatin, lactose, amylose, magnesium stearate, talc, silic acid, hydroxymethyl-cellulose, etc. The pharmaceutical compositions can be sterilized and if desired mixed with auxiliary agents, e.g. lubricants, preservatives, stabilizers, wetting agents, emulsifiers, buffers, colorings, flavoring and the like which deleteriously react with the active compound(s).

For parenteral application, particularly suitable are solutions, preferably oily or aqueous solutions as well as suspensions, emulsions, or implants, including suppositories.

For enteral application, particularly suitable are tablets, dragees or capsules having talc and/or carbohydrate carrier binder or the like. A syrup, elixir or the like also can be employed. Sustained release compositions can be formulated including those wherein the active component is protected with differentially degradable coatings, e.g., by microencapsulation, multiple coatings, etc.

It often will be preferable to use an optically active or enantiomerically enriched mixture of a chiral compound of the invention for a given therapeutic application. As used herein, the term “enantiometrically enriched” or similar term typically refers to a compound mixture that is at least approximately 70 mole %, 80 mole %, 85 mole % or 90 mole % of a single stereoisomer, and preferably a compound mixture that contains approximately at least about 92 mole %, 95 mole %, 97 mole %, 98 mole %, 99 mole % or 100% of a single enantiomer of the compound.

It will be appreciated that the actual preferred amounts of active compounds used in a given therapy will vary according to the specific compound being utilized, the particular compositions formulated., the mode of application, the particular site of administration, etc. Optimal administration rates for a given protocol of administration can be readily ascertained by those skilled in the art using conventional dosage determination tests conducted with regard to the foregoing guidelines. In general, a suitable effective dose of a compound of any of Formulae I through VI, particularly when using the more potent compounds of Formulae I through VI, is from about 10 ng/kg or recipient to 300 mg/kg or recipient, preferably 0.1 to 100 mg/kg per day, more typically 0.5 to 25 mg per kilogram body weight of the recipient per day. A typical topical dosage will range from 0.01 to 3% wt/wt in a suitable carrier.

All documents mentioned herein are incorporated herein by reference. The following non-limiting examples are illustrative of the invention.

In the following examples, compounds are further referenced to the structures set forth in Scheme 1 above, as well as the specifically preferred compounds referenced above as compounds 2A, 2B, 3, 4A, 4B, 5, 6A and 6B.

### EXAMPLE 1

### Synthesis of 2-Iodo-4-Trifluoromethylphenol (Scheme 1: 8)

4-Trifluoromethylphenol (25 g, 154 mmol; Compound 7 in the above Scheme 1) was taken in DMF (100 mL) and to the resulting suspension was added NaI (30 g, 200 mmol) and stirred for 15 min. Chloramine-T (38.5 g, 169 mmol) was added to the reaction mixture and it was stirred for 6 h at room temperature. The reaction mixture was acidified by the addition of 1N HCl and was poured into ice-water mixture (4 L). The resulting suspension was stirred for some time, the light brown solid was filtered and purified on a flash column (25% ethyl acetate in hexanes), to give 22.2 g (50%) of the title compound (Compound 8).

### EXAMPLE 2

### Synthesis of [5-(Trifluoromethyl)benzo[d]furan-2-yl]methan-1-ol (Scheme 1: 9).

Propargyl alcohol (9 mL, 154 mmol), Compound 8 as prepared in Example 1 above (22.2 g, 77 mmol) and Cu₂O (6.9 g, 48 mmol) were taken in pyridine (40 mL) and the resulting mixture was refluxed overnight. The mixture was then cooled to room temperature, diluted with toluene (500 mL) and filtered through celite. The filtrate was concentrated on a rotary evaporator and the residue was subjected to a flash column (30% ethyl acetate in hexanes) to give compound 9 (11.6 g, 70%).

### EXAMPLE 3

### Synthesis of 5-(Trifluoromethyl)benzo[b]furan-2-carbaldehyde (Scheme 1: 10).

PCC (17.24 g, 80 mmol) was taken in methylene chloride (300 mL) and the resulting suspension was stirred for 15 min. To that mixture was added Compound 9 as prepared in Example 2 above (11.6 g, 54 mmol) and the reaction mixture was stirred overnight. The mixture was poured into vigorously stirred ether (2 L) and resulting suspension was filtered through celite. The filtrate was concentrated on a rotary evaporator and the residue was passed thought a pad of silica gel (50% ethyl acetate in hexanes) to obtain 9.2 g (80%) of the title compound (Compound 10).

### EXAMPLE 4

### Synthesis of Tert-Butyl 4-oxo-4-[5-(trifluoromethyl)benzo[d]furan-2-yl]butanoate (Scheme 1: 11).

tert-Butyl acrylate (6.3 mL, 43 mmol), Compound 10 as prepared in Example 3 above (9.2 g, 43 mmol) and ETB (4.3 g, 17.2 mmol) were dissolved in dry DMF (20 mL). To this solution was added triethylamine (12 mL) with stirring. The reaction mixture was heated at 50° C. overnight (the color changed to dark green and then to dark brown within 3-4 min. of heating). The reaction mixture was acidified by adding 1 N HCl (1.5 L) and extracted with ethyl acetate (3×1 L). Combined organic extracts were washed with water (2×1.5 L) and brine (1 L) and dried over sodium sulfate. The ethyl acetate solution was concentrated on a rotary evaporator and the residue was crystallized from MeOH/H₂O to yield 7.35 g (50%) of the title compound (Compound 11).

### EXAMPLE 5

### Synthesis of (5S)-5-[5-(Trifluoromethyl)benzo[d]furan-2-yl]-3,4,5-trihydrofuran-2-one (Scheme 1: 12).

A solution of Compound 11 as prepared in Example 4 above (6.85 g, 20 mmol) in dry THF (5 mL) was added, dropwise, to a precooled (0° C.) solution of (−)-DIP-chloride (10.9 g, 34 mmol) in THF (10 mL) with stirring under dry argon. The resulting solution was stirred at the same temperature for 1 h and then it was allowed to stand at 0-5° C. for 24 h. Maintaining the temperature at 0° C., with stirring, water (8 mL) was added dropwise followed by methanol (20 mL) and 5M solution of NaOH until strongly basic (˜20 mL). The reaction mixture was stirred at room temperature for 2 hours. The resulting mixture was concentrated on a rotavap to remove THF and MeOH. The residue was diluted with water (200 mL) and then was washed with ether (3×300 mL). The aqueous layer was acidified with 6N HCl and was extracted with toluene (3×300 mL). The combined toluene extracts were washed with water (300 mL) and brine (300 mL), dried over sodium sulfate and concentrated on a rotavap to approximately half the volume. To the resulting solution was added PPTS (70 mg) and it was refluxed under a Dean-Stark trap for 6 h. It was cooled, washed with water (3×300 mL), dried over sodium sulfate and concentrated to give 4 g (70%) of the title compound (Compound 12).

### EXAMPLE 6

### Synthesis of (5S)-5-[5-(Trifluoromethyl)benzo[d]furan 2-yl]oxolan-2-ol (Scheme 1: 13).

A solution of Compound 12 as prepared in Example 5 above (2.5 g, 8.75 mmol) in dry methylene chloride (100 mL) was cooled to −78° C. and, with stirring under argon, DIBAH (9 mL of 1.5 M solution in toluene, 13.1 mmol) was added to it dropwise. Stirring was continued at −78° C. for 6 h and then a saturated aqueous solution of Na—K-tartarate (250 mL) was added. The cooling bath was removed and the stirring was continued overnight. The organic layer was separated, washed with water (2×250 ML) and brine (200 mL), dried over sodium sulfate and rotavaped to give the title compound, (5S) 5-[5-(trifluoromethyl)benzo[d]furan-2-yl]oxolan-2-ol (Compound 13, 2.5 g, 100%).

### EXAMPLE 7

### Synthesis of 1-{(5S)-5-[5-(Trifluoromethyl)benzo[d]furan-2-yl]oxolan-2-yloxy}-1,1,2,2-tetramethy-1-silapropane (Scheme 1: 14).

A solution of (5S)-5-[5-(trifluoromethyl)benzo[d]furan-2-yl]oxolan-2-ol (Compound 13, 2.5 g, 8.7 mmol) and imidazole (7.69 g, 11.31 mmol) in dry methylene chloride (20 mL) was cooled to 0° C. and t-butyldimethylsilyl chloride (1.57 g, 10.4 mmol) was added to the mixture. The resulting solution was stirred under dry argon overnight at room temperature. It was then diluted with ethyl acetate (300 mL) and was washed with water (3×250 mL) and brine (100 mL), dried over sodium sulfate and the solvent removed using a rotary evaporator to give 3.5 g (100%) of the title compound (Compound 14).

### EXAMPLE 8

### Synthesis of 1-(4-{(2S,5S)-5-[5-(Trifluoromethyl)benzo[d]furan-2-yl}oxolan-2-yl}but-3-ynyloxy)-1,1,2,2-tetramethyl-1-silapropane (Scheme 1: 15) and 1-(4-{(2S,5R)-5-[5-(trifluoromethyl)benzo[d]furan-2-yl}loxolan-2-yl}but-3-ynyloxy)-1,1,2,2-tetramethyl-1-silapropane (Scheme 1: 16).

1-{(5S)-5-[5-(Trifluoromethyl)benzo[d]furan-2-yl]oxolan-2-yloxy}-1,1,2,2-tetramethy-1-silapropane (Scheme 1: 14) (3.5 g, 8.7 mmol) were taken in 10 mL of dry methylene chloride (degassed by bubbling argon prior to use). This solution was cooled to −40° C. and, while stirring at the same temperature under dry argon, trimethylsilyl bromide (1.4 mL, 10.4 mmol) was added dropwise. The stirring was continued for an additional 3 hours.

In a separate flask, 3-tert-butyldimethylsilyloxy-but-1-yne (1.9 g, 10.4 mmol) was taken in dry THF (10 mL). The solution was cooled to −78° C. and, while stirring at the same temperature under dry argon, n-butylithium (4.2 mL of 2.5M solution in hexane, 10.4 mmol) was added dropwise. The stirring was continued for an additional 0.5 h. The resulting solution was added dropwise, through a cannula to the stirred solution of the 2-bromotetrahydrofuran (made above) at −78° C. The stirring was continued at −78° C. for additional 2 h. The reaction was quenched by adding saturated aqueous solution of ammonium chloride (50 mL). The reaction mixture was warmed to room temperature and was extracted with ethyl acetate (3×100 mL). The combined extracts were washed with brine, dried over sodium sulfate and the solvent removed using a rotary evaporator. The residue was subjected to flash column chromatography (eluent, 10% ethyl acetate in hexane) to obtain two components. From the proton NMR analysis, the less polar one was identified as the trans-(2S,5S) isomer (Compound 15, 800 mg, 41%)) and the more polar component was assigned to be the cis-(2R,5S) isomer (Compound 16, 800 mg, 41%).

### EXAMPLE 9

### Synthesis of 4-{(2S,5S)-5-[5-(Trifluoromethyl)benzo[d]furan-2-yl]oxolan-2-yl}but-3-yn-1-ol (Scheme 1: 17).

The trans-(2S,5S) isomer 15 (Compound 15, 800 mg 1.76 mmol) was taken in 25 mL of THF. The solution was cooled to 0° C. and TBAF (5.3 mL of 1M solution in THF, 5.3 mmol) was added to it. The resulting solution was stirred at 0° C. for 1 h and then rotavaped to remove THF. The residue was taken in ethyl acetate (100 mL), washed with water (3×200 mL, added 10 mL of brine each time to separate layers) followed by brine (50 mL), dried over sodium sulfate and rotavaped to obtain 600 mg (100%) of the title compound (Compound 17).

### EXAMPLE 10

### Synthesis of N-(4-{(2S,5S)-5-[5-(Trifluoromethyl)benzo[d]furan-2-yl]oxolan-2 yl}but-3-ynyl)phenoxycarbonylamino Phenoxyformate (Scheme 1: 18).

Triphenylphosphine (555 mg, 2.12 mmol), Compound 17 of Example 9 (600 mg, 1.76 mmol) and N,O-bis(phenoxycarbonyl)hydroxylamine (545 mg, 2.12 mmol) were dissolved in dry THF (10 mL). The solution was cooled to 0° C. and with stirring under dry argon was added diisopropylazodicarboxylate (420 μL, 2.12 mmol) dropwise. The stirring was continued for 30 min. at the same temperature. The solvent was evaporated on a rotavap and the residue was subjected to flash column chromatography (eluent, 30% ethyl acetate in hexane) to give 920 mg (90%) of the title compound (Compound 18).

### EXAMPLE 11

### Synthesis of N-(4-{(2S,5S)-5-[5-(Trifluoromethyl)benzo[d]furan-2-yl]oxolan-2-yl)but-3-ynyl) Amino-N-hydroxyamide (Scheme 1: 1A).

The phenoxyformate compound of Example 10 (Compound 18, 920 mg, 1.59 mmol) was taken in a high pressure tube as a solution in methanol (50 mL). The solution was cooled to −78° C. Approximately 5 mL of ammonia was condensed into this tube. The tube was sealed and was allowed to slowly warm to the room temperature. Then it was left stirring at rt overnight. The pressure was released very slowly and the tube was left open for 1 h. The reaction mixture was transferred into a flask and concentrated and the residue was subjected to a flash column (eluent, 3% methanol in methylene chloride) to give 465 mg (74%) of the title compound, Compound 1A.

### EXAMPLE 12

### Synthesis of 4-{(2S,5R)-5-[5-(Trifluoromethyl)benzo[d]furan-2-yl]oxolan-2-yl} but-3-yn-1-ol (Scheme 1: 13).

Starting with cis isomer 16 (Scheme 1) (210 mg, 0.44 mmol), following the same procedure as detailed for the preparation of Compound 17, 151 mg (100%) of the title compound (Compound 19) was obtained.

### EXAMPLE 14

### Synthesis of N-(4-{(2S,5R)-5-[5-(Trifluoromethyl)benzo[d]furan-2-yl]oxolan-2-yl}but-3-ynyl)phenoxycarbonylamino Phenoxyformate (Scheme 1: 20).

Starting with Compound 19 (151 mg, 0.44 mmol), following the same procedure as for the preparation of Compound 18, 204 mg (80%) of the title compound (Compound 20) was obtained.

### EXAMPLE 15

### Synthesis of N-(4-{(2S,5R)-5-[5-(Trifluoromethyl)benzo[d]furan-2-yl]oxolan-2-yl}but-3-ynyl) Amino-N-hydroxyamide (Scheme 1: 1B).

Starting with Compound 20 (204 mg, 0.35 mmol), following the same procedure as for the preparation of Compound 1A, 37 mg (27%) of the title Compound 1B was obtained.

### EXAMPLE 16

### Inhibition of Leukotriene B4 Production in lonophore-Stimulated Human Whole Blood

Human blood was drawn into heparinized blood collection tubes, and aliquoted in 1 ml portions into 1.5 ml microfuge tubes. Test compound (5 ml) of varying concentrations, dissolved in DMSO, was added to the blood sample and incubated for 15 minutes at 37° C. Calcium ionophore (5 ml) in DMSO was added to a final concentration of 50 mM, and the samples were incubated for 30 minutes at 37° C. Samples are then centrifuged at 1100×g (2500 rpm, H1000B rotor, in a Sorvall centrifuge) for 10 minutes at 4° C. Supernatant (100 ml) was transferred into 1 1.5 ml microfuge tube, 400 ml of cold methanol added, and proteins precipitated on ice for 30 minutes. The samples were centrifuged at 110×g for 10 minutes at 4° C., and the supernatant assayed for LTB₄ using a commercially available EIA kit (Cayman Chemical) according to manufacturer's specifications. The following IC₅₀ results (nM) were obtained for the specified compounds having the structures as indicated for the corresponding compound number.

| Compound No. | HWB IC₅₀ (nM) |
|---|---|
| 2A | 112 |
| 2B | 878 |
| 3 | 197 |
| 4A | 177 |
| 4B | 298 |
| 5 | 533 |
| 6A | 101 |
| 6B | 388 |

The invention has been described in detail including preferred embodiments thereof. However, it will be understood that those skilled in the art, upon consideration of this disclosure, may make modifications and improvements thereon without departing from the spirit and scope of the invention as set forth in the following claims.

What is claimed is:

1. A compound of the formula following Formula I: 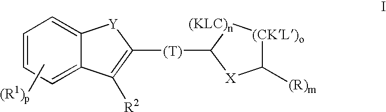
wherein each R, R¹, R², K, L, K′, and L′ is independently hydrogen, halogen, cyano, hydroxyl, optionally substituted alkyl, optionally substituted alkenyl, optionally substituted alkynyl, optionally substituted alkoxy, optionally substituted alkylthio, optionally substituted alkylsulfinyl, optionally substituted alkylsulfonyl, optionally substituted aminoalkyl, optionally substituted alkanoyl, optionally substituted carbocyclic aryl, optionally substituted aralkyl;
X is O, S, S(O), S(O)₂, NH, substituted N or a chemical bond;
T is a chemical bond, optionally substituted alkylene, optionally substituted alkenylene, optionally substituted alkynylene, optionally substituted heteroalkynylene, or a hetero atom, or NR;
Y is O, S, S(O), S(O)₂ or a chemical bond;
m is 0, 1 or 2;
o and n are each integers of 0 or greater, the sum of o and n being from 1 to about 8; and
p is an integer of from 0 to 4; and pharmaceutically acceptable salts thereof.

2. A compound of claim 1 wherein K, L, K′, and L′ are each hydrogen.

3. A compound of claim 1 or 2 wherein R comprises one or more N, O or S atoms and m is 1.

4. A compound of claim 1 wherein the compound is of the following Formula II: 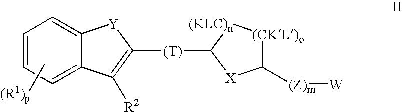
wherein each R¹, R², K, L, K′ and L′ is independently hydrogen, halogen, cyano, hydroxyl, optionally substituted alkyl, optionally substituted alkenyl, optionally substituted alkynyl, optionally substituted alkoxy, optionally substituted alkylthio, optionally substituted alkylsulfinyl, optionally substituted alkylsulfonyl, optionally substituted aminoalkyl, optionally substituted alkanoyl, optionally substituted carbocyclic aryl, optionally substituted aralkyl;
X is O, S, S(O), S(O)₂, NH, substituted N or a chemical bond;
T is a chemical bond, optionally substituted alkylene, optionally substituted alkenylene, optionally substituted alkynylene, optionally substituted heteroalkynylene, or a hetero atom, or NR;
Y is O, S, S(O), S(O)₂ or a chemical bond;
Z is O, S, S(O), S(O)₂, NR¹ or a chemical bond;
W is —AN(OM)C(O)N(R¹R²), —N(OM)C(O)N(R¹R²), —AN(R)C(O)N(OM)R¹, —N(R)C(O)N(OM)R¹, —AN(OM)C(O)R¹, —N(OM)C(O)R¹, —AC(O)N(OM)R¹, —C(O)N(OM)R¹, —C(O)NHA, where A is optionally substituted alkyl, optionally substituted alkenyl, optionally substituted alkynyl, optionally substituted carbocyclic aryl, and where one or more carbon atoms can be optionally replaced with O, substituted O, N, substituted N, S, or substituted S; and M is hydrogen, a pharmaceutically acceptable cation, or a metabolically cleavable leaving group;
p is an integer of from 0 to 4;
o and n are each integers of 0 or greater, the sum of o and n being from 1 to about 8;
m is 0 or 1; and pharmaceutically acceptable salts thereof.

5. A compound of claim 4 wherein K, L, K′, and L′ are each hydrogen.

6. A compound of any one of claims 1 through 5 wherein Y is oxygen.

7 A compound of claim 4 according to formula III: 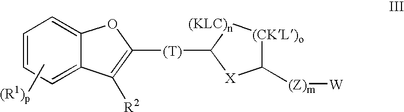
wherein each R¹, R², K, L, K′ and L′ is independently hydrogen, halogen, cyano, hydroxyl, optionally substituted alkyl, optionally substituted alkenyl, optionally substituted alkynyl, optionally substituted alkoxy, optionally substituted alkylthio, optionally substituted alkylsulfinyl, optionally substituted alkylsulfonyl, optionally substituted aminoalkyl, optionally substituted alkanoyl, optionally substituted carbocyclic aryl, optionally substituted aralkyl;
X is O, S, S(O), S(O)₂, NH, substituted N or a chemical bond;
T is a chemical bond, optionally substituted alkylene, optionally substituted alkenylene, optionally substituted alkynylene, optionally substituted heteroalkynylene, or a hetero atom, or NR;
Z is O, S, S(O), S(O)₂, NR¹ or a chemical bond;
W is —AN(OM)C(O)N(R¹R²), —N(OM)C(O)N(R¹R²), —AN(R)C(O)N(OM)R¹, —N(R)C(O)N(OM)R¹, —AN(OM)C(O)R¹, —N(OM)C(O)R¹, —AC(O)N(OM)R¹, —C(O)N(OM)R¹, —C(O)NHA, where A is optionally substituted alkyl, optionally substituted alkenyl, optionally substituted alkynyl, optionally substituted carbocyclic aryl, and where one or more carbon atoms can be optionally replaced with O, substituted O, N, substituted N, S, or substituted S; and M is hydrogen, a pharmaceutically acceptable cation, or a metabolically cleavable leaving group;
p is an integer of from 0 to 4;
o and n are each integers of 0 or greater, the sum of o and n being from 1 to about 8;
m is 0 or 1; and pharmaceutically acceptable salts thereof.

8. A compound of claim 1 wherein the compound is of the following Formula IV: 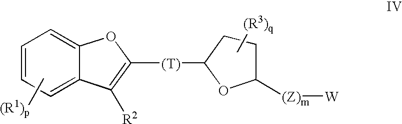
wherein each R¹, R², and R³ is independently hydrogen, halogen, cyano, hydroxyl, optionally substituted alkyl, optionally substituted alkenyl, optionally substituted alkynyl, optionally substituted alkoxy, optionally substituted alkylthio, optionally substituted alkylsulfinyl, optionally substituted alkylsulfonyl, optionally substituted aminoalkyl, optionally substituted alkanoyl, optionally substituted carbocyclic aryl, optionally substituted aralkyl;
Z is O, S, S(O), S(O)₂, NR¹ or a chemical bond;
W is —AN(OM)C(O)N(R¹R²), —N(OM)C(O)N(R¹R²), —AN(R)C(O)N(OM)R¹, —N(R)C(O)N(OM)R¹, —AN(OM)C(O)R¹, —N(OM)C(O)R¹, —AC(O)N(OM)R¹, —C(O)N(OM)R¹, —C(O)NHA, where A is optionally substituted alkyl, optionally substituted alkenyl, optionally substituted alkynyl, optionally substituted carbocyclic aryl, and where one or more carbon atoms can be optionally replaced with O, substituted O, N, substituted N, S, or substituted S; and M is hydrogen, a pharmaceutically acceptable cation, or a metabolically cleavable leaving group;
m is 0 or 1;
p is an integer of from 0 to 4; and
q is 0, 1 or 2; and pharmaceutically acceptable salts thereof.

9. A compound of claim 1 wherein the compound is of the following Formula V: 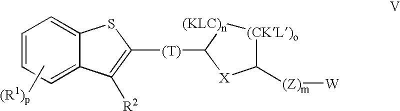
wherein each R¹, R², K, L, K′ and L′ is independently hydrogen, halogen, cyano, hydroxyl, optionally substituted alkyl, optionally substituted alkenyl, optionally substituted alkynyl, optionally substituted alkoxy, optionally substituted alkylthio, optionally substituted alkylsulfinyl, optionally substituted alkylsulfonyl, optionally substituted aminoalkyl, optionally substituted alkanoyl, optionally substituted carbocyclic aryl, optionally substituted aralkyl;
X is O, S, S(O), S(O)₂, NH, substituted N or a chemical bond;
T is a chemical bond, optionally substituted alkylene, optionally substituted alkenylene, optionally substituted alkynylene, optionally substituted heteroalkynylene, or a hetero atom, or NR;
Z is O, S, S(O), S(O)₂, NR¹ or a chemical bond;
W is —AN(OM)C(O)N(R¹R²), —N(OM)C(O)N(R¹R²), —AN(R)C(O)N(OM)R¹, —N(R)C(O)N(OM)R¹, —AN(OM)C(O)R¹, —N(OM)C(O)R¹, —AC(O)N(OM)R¹, —C(O)N(OM)R¹, —C(O)NHA, where A is optionally substituted alkyl, optionally substituted alkenyl, optionally substituted alkynyl, optionally substituted carbocyclic aryl, and where one or more carbon atoms can be optionally replaced with O, substituted O, N, substituted N, S, or substituted S; and M is hydrogen, a pharmaceutically acceptable cation, or a metabolically cleavable leaving group;
p is an integer of from 0 to 4;
o and n are each integers of 0 or greater, the sum of o and n being from 1 to about 8;
m is 0 or 1; and pharmaceutically acceptable salts thereof.

10. A compound of claim 9 wherein K, L, K′, and L′ are each hydrogen.

11. A compound of claim 1 wherein the compound is of the following Formula VI: 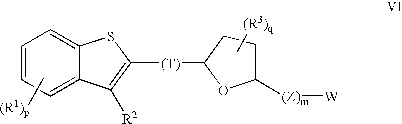
wherein each R¹, R², and R³ is independently hydrogen, halogen, cyano, hydroxyl, optionally substituted alkyl, optionally substituted alkenyl, optionally substituted alkynyl, optionally substituted alkoxy, optionally substituted alkylthio, optionally substituted alkylsulfinyl, optionally substituted alkylsulfonyl, optionally substituted aminoalkyl, optionally substituted alkanoyl, optionally substituted carbocyclic aryl, optionally substituted aralkyl;
Z is O, S, S(O), S(O)₂, NR¹ or a chemical bond;
W is —AN(OM)C(O)N(R¹R²), —N(OM)C(O)N(R¹R²), —AN(R)C(O)N(OM)R¹, —N(R)C(O)N(OM)R¹, —AN(OM)C(O)R¹, —N(OM)C(O)R¹, —AC(O)N(OM)R¹, —C(O)N(OM)R¹, —C(O)NHA, where A is optionally substituted alkyl, optionally substituted alkenyl, optionally substituted alkynyl, optionally substituted carbocyclic aryl, and where one or more carbon atoms can be optionally replaced with O, substituted O, N, substituted N, S, or substituted S; and M is hydrogen, a pharmaceutically acceptable cation, or a metabolically cleavable leaving group;
m is 0 or 1;
p is an integer of from 0 to 4;
q is 0, 1 or 2; and pharmaceutically acceptable salts thereof.

12. A compound of any one of claims 1 through 11 wherein each R¹ is independently selected from the group consisting of hydrogen, halogen, haloalkyl, haloalkoxy, and optionally substituted carboxylic aryloxy.

13. A compound of any one of claims 1 through 11 wherein p is 1 and each R1 is independently 4-trifluoromethyl, 4-halo or 4-alkoxy.

14. A compound of any one of claims 1 through 13 wherein T is a chemical bond.

15. A compound of any one of claims 1 through 14 wherein Z is a chemical bond.

16. A compound of any one of claims 1 through 15 wherein R and W is —(CH₂)₂CH(CH₃)H— or —C≡C—CH₂(C₁₋₆alkyl)-.

17. A compound of any one of claims 4 through 15 wherein W is selected from the group consisting of:

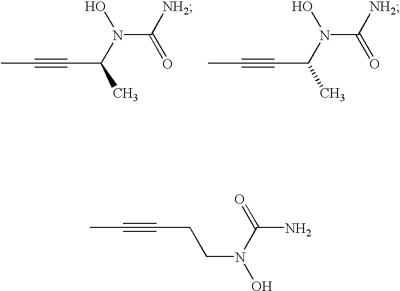

18. A compound of any one of claims 1 through 17 wherein an enatiomeric excess of one stereoisomer is present.

19. A compound of claim 1 that is N-(4-(-5-[5-(trifluoromethyl)benzo [d]furan-2-yl]oxolan-2-yl)but-3-ynyl) amino-N-hydroxyamide and pharmaceutically acceptable salts thereof.

20. A compound of claim 1 that is N-(4-((2S,5S)-5-[5-(trifluoromethyl)benzo[d]furan-2-yl]oxolan-2-yl)but-3-ynyl) or N-(4-((2S,5R)-5-[5-(trifluoromethyl)benzo[d]furan-2-yl]oxolan-2-yl)but-3-ynyl) amino-N-hydroxyamide, or a pharmaceutically acceptable salt of said compounds.

21. A pharmaceutical composition comprising a compound of any one of claims 1 through 20 and a pharmaceutically acceptable carrier.

22. A method of treating a disorder or disease associated with 5-lipoxygenase, comprising administering to a subject suffering from or susceptible to such a disease or disorder an effective amount of a compound or composition of any one of claims 1 through 21.

23. A method of treating a immune, allergic or cardiovascular disorder or disease, comprising administering to a subject suffering from or susceptible to such a disease or disorder an effective amount of a compound of any one of claims 1 through 20.

24. A method of treating a immune, allergic or cardiovascular disorder or disease, comprising administering to a subject suffering from or susceptible to such a disease or disorder an effective amount of a compound of any one of claims 1 through 20.

25. A method of treating a immune, allergic or cardiovascular disorder or disease, comprising administering to a subject suffering from or susceptible to such a disease or disorder an effective amount of a compound of any one of claims 1 through 20.

26. A method for treating a disorder or disease of inflammation, hypertension, skeletal-muscular disorders, osteoarthritis, gout, asthma, lung edema, adult respiratory distress syndrome, pain, aggregation of platelets, shock, rheumatoid arthritis, psoriatic arthritis, psoriasis, inflammatory bowel disease, chronic obstructive pulmonary disease, autoimmune uveitis, allergic encephalomyelitis, systemic lupus erythematosis, acute necrotizing hemmorrhagic encephalopathy, idiopathic thrombocytopenia, polychondritis, chronic active hepatitis, idiopathic sprue, Crohn's disease, Graves ophthalmopathy, primary biliary cirrhosis, uveitis posterior, interstitial lung fibrosis, or allergic asthma, comprising administering to a subject suffering from or susceptible to the disorder an effective amount of a compound or composition of any one of claims 1 though 20.

27. A method of any one of claims 22 through 26 wherein the subject is a human.
